(19) United States
(12) Patent Application Publication
(10) Pub. No.: US 2026/0368752 A1
(43) Pub. Date: Nov. 19, 2026

(54) AUTHENTICITY DETERMINATION SUPPORT INFORMATION CREATION ASSISTING DEVICE

(71) Applicant: NEC Corporation, Tokyo (JP)

(72) Inventor: Eiki ISHIDERA, Tokyo (JP)

(73) Assignee: NEC Corporation, Tokyo (JP)

(21) Appl. No.: 19/137,726

(22) PCT Filed: Dec. 20, 2022
(86) PCT No.: PCT/JP2022/046774

§ 371 Date: Jun. 11, 2025

(51) Int. Cl. G06Q 30/018; G06F 16/58; G06T 7/13; G06T 7/194; G06V 10/46; G06V 30/10; G16H 20/10

(52) U.S. Cl. CPC G06Q 30/018; G06F 16/5866; G06T 7/13; G06T 7/194; G06V 10/46; G06V 30/10; G16H 20/10

(57) ABSTRACT

An authenticity determination support information creation assisting device includes: object image acquiring means for acquiring an image of a surface showing the pharmaceutical name of a pharmaceutical package as an object image; frontalized image generating means for generating a frontalized image by extracting a region of the surface from the object image and performing trapezoidal correction; pharmaceutical name recognizing means for recognizing the pharmaceutical name by performing character recognition from the frontalized image; authentic pharmaceutical name retrieving means for retrieving a similar pharmaceutical name as an authentic pharmaceutical name from an authorized pharmaceutical name list by using the pharmaceutical name; and authentic product image retrieving means for retrieving an authentic product image from image information in which the authentic pharmaceutical name and the authentic product image showing the pharmaceutical name of an authentic pharmaceutical package are associated with each other, by performing image retrieval using the authentic pharmaceutical name.

### TECHNICAL FIELD

The present invention relates to an authenticity determination support information creation assisting device, an authenticity determination support information creation assisting method, and a storage medium.

### BACKGROUND ART

In countries in Africa or the like, it is said that about 40% of pharmaceutical products distributed in the market are fake drugs, which has become a serious social problem. Many fake drugs are adulterated, and there are cases where users can determine whether or not they are fake drugs by simply comparing the image of a pharmaceutical package with an authentic product.

Therefore, in some countries such as Kenya, experts create and distribute leaflets explaining information for supporting authenticity determination by the users in illustrations and sentences when fake drugs are distributed.

On the other hand, Patent Literature 1 describes displaying information for supporting authenticity determination by the users. To be specific, an authenticity check point related to a product or the like is acquired from a database based on a bar code or the like attached to the product or the like, and displayed on the screen of a mobile terminal. This allows a user to grasp important points in determining whether the product is fake or genuine, that is, determining authenticity.

In addition, the following documents also describe the techniques related to the present invention.

Patent Literature 2 describes a prescription order processing system for preventing incorrect prescription of a drug based on a prescription order, in which an entered drug name is searched in a database of similar drug names and presence or absence of similar drug names is examined.

Patent Literature 3 describes performing character recognition on an image of a label of an over-the-counter drug, extracting the text of the product name and ingredients, and searching a drug database.

Patent Literature 4 describes a review support system that supports a review of a preparation performed based on prescription data, in which a difference between a photographed image of a tablet and a positive image of the tablet is detected and displayed when the result of the image review processing is displayed.

### CITATION LIST

### Patent Literature

Patent Literature 1: JP 2003-150488 A
Patent Literature 2: JP 2006-163541 A
Patent Literature 3: JP 2016-151876 A
Patent Literature 4: JP 2020-075172 A

### SUMMARY OF INVENTION

### Technical Problem

With respect to pharmaceutical products, it is desirable to store information that supports users in making an authenticity determination in a database, and provide the information to a user who needs the information, so as to allow the user to determine whether the pharmaceutical product is genuine or fake. However, in order to do so, experts with specialized knowledge on the authenticity determination on pharmaceutical products must prepare information in advance for supporting authenticity determination by the users. Creating such information entirely by manual takes a great deal of time and cost.

An object of the present invention is to provide an authenticity determination support information creation assisting device that solves the aforementioned problem, that is, it takes a great deal of time and cost to create information for supporting authenticity determination on a pharmaceutical product by the users.

### Solution to Problem

An authenticity determination support information creation assisting device, according to one aspect of the present invention, is configured to include
an object image acquiring means for acquiring an image obtained by capturing a surface showing a pharmaceutical name of a pharmaceutical package as an object image,
a frontalized image generating means for generating a frontalized image by extracting a region of the surface from the acquired object image and performing trapezoidal correction,
a pharmaceutical name recognizing means for recognizing the pharmaceutical name by performing character recognition from the generated frontalized image,
an authentic pharmaceutical name retrieving means for retrieving a similar pharmaceutical name as an authentic pharmaceutical name from an authorized pharmaceutical name list by using the recognized pharmaceutical name, and
an authentic product image retrieving means for retrieving an authentic product image from image information in which the authentic pharmaceutical name and the authentic product image obtained by capturing a surface showing a pharmaceutical name of an authentic pharmaceutical package are associated with each other, by performing image retrieval using the retrieved authentic pharmaceutical name as a keyword.

An authenticity determination support information creation assisting method, according to another aspect of the present invention, is configured to include
acquiring an image obtained by capturing a surface showing a pharmaceutical name of a pharmaceutical package as an object image,
generating a frontalized image by extracting a region of the surface from the acquired object image and performing trapezoidal correction,
recognizing the pharmaceutical name by performing character recognition from the generated frontalized image,
retrieving a similar pharmaceutical name as an authentic pharmaceutical name from an authorized pharmaceutical name list by using the recognized pharmaceutical name, and
retrieving an authentic product image from image information in which the authentic pharmaceutical name and the authentic product image obtained by capturing a surface showing a pharmaceutical name of an authentic pharmaceutical package are associated with each other, by performing image retrieval using the retrieved authentic pharmaceutical name as a keyword.

A computer-readable medium, according to another aspect of the present invention, is configured to store thereon a program for causing a computer to execute processing to
acquire an image obtained by capturing a surface showing a pharmaceutical name of a pharmaceutical package as an object image,
generate a frontalized image by extracting a region of the surface from the acquired object image and performing trapezoidal correction,
recognize the pharmaceutical name by performing character recognition from the generated frontalized image,
retrieve a similar pharmaceutical name as an authentic pharmaceutical name from an authorized pharmaceutical name list by using the recognized pharmaceutical name, and
retrieve an authentic product image from image information in which the authentic pharmaceutical name and the authentic product image obtained by capturing a surface showing a pharmaceutical name of an authentic pharmaceutical package are associated with each other, by performing image retrieval using the retrieved authentic pharmaceutical name as a keyword.

An authenticity determination support system, according to another aspect of the present invention, is an authenticity determination support system for supporting authenticity determination on a determination object by a user. The system is configured to include
a database that is created by using the authenticity determination support information creation assisting device according to any of claims 1 to 5 and in which authenticity determination support information is stored, the authenticity determination support information including an image obtained by capturing a genuine pharmaceutical package and an image obtained by capturing a fake of the genuine pharmaceutical package,
an acquiring means for acquiring an image obtained by capturing the determination object from a user terminal,
a matching means for matching the image obtained by capturing the determination object against an image in the authenticity determination support information stored in the database, and
a presenting means for, when the authenticity determination support information including an image matching the image obtained by capturing the determination object is detected as a result of the matching, allowing the detected authenticity determination support information to be displayed on a screen of the user terminal.

### Advantageous Effects of Invention

With the aforementioned configurations, the present invention can reduce the time and cost when experts create information for supporting authenticity determination on a pharmaceutical product by the users.

### BRIEF DESCRIPTION OF DRAWINGS

FIG. 1 is a block diagram of an authenticity determination support information creation assisting device according to a first example embodiment of the present invention.

FIG. 2 illustrate an example of information stored in an authenticity determination support information database in the first example embodiment of the present invention.

FIG. 3 is a flowchart illustrating an example of operation of the authenticity determination support information creation assisting device according to the first example embodiment of the present invention.

FIG. 4 is a schematic diagram illustrating an example of authenticity determination support information created using the authenticity determination support information creation assisting device according to the first example embodiment of the present

FIG. 5 is a schematic diagram illustrating another example of authenticity determination support information created using the authenticity determination support information creation assisting device according to the first example embodiment of the present invention.

FIG. 6 is a block diagram illustrating an example configuration of a frontalized image generating unit in the first example embodiment of the present invention.

FIG. 7 is a block diagram illustrating an example configuration of a pharmaceutical name recognizing unit in the first example embodiment of the present invention.

FIG. 8 is a block diagram illustrating an example configuration of a difference detecting unit in the first example embodiment of the present invention.

FIG. 9 is a diagram illustrating an example of a CNN used by the difference detecting unit in the first example embodiment of the present invention.

FIG. 10 is a block diagram of an authenticity determination support system according to a second example embodiment of the present invention.

FIG. 11 is a block diagram illustrating an example of a server terminal in the second example embodiment of the present invention.

FIG. 12 is a flowchart illustrating an example of operation of the server terminal when a support request is sent from a user terminal in the second example embodiment of the present invention.

FIG. 13 is a flowchart illustrating an example of operation of a server terminal 10 when authenticity determination support information is sent from an expert terminal in the second example embodiment of the present invention.

FIG. 14 is a block diagram of an authenticity determination support information creation assisting device according to a third example embodiment of the present invention.

### DESCRIPTION OF EMBODIMENTS

### First Example Embodiment

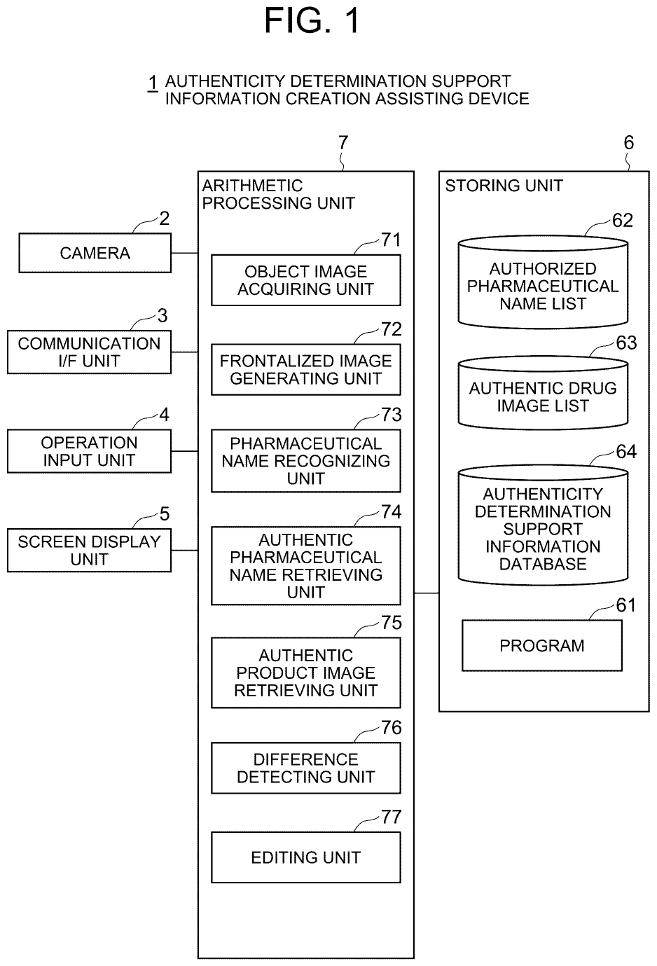

FIG. 1 is a block diagram of an authenticity determination support information creation assisting device 1 according to a first example embodiment of the present invention. The authenticity determination support information creation assisting device (hereinafter sometimes simply referred to as an assisting device) 1 is a device used by experts who create authenticity determination support information, and a pharmaceutical package that is independently subject to transaction is an object of authentication determination. For example, primary packaging that comes into direct contact with a pharmaceutical product such as a container or a PTP sheet and an outside container or an outside wrapper that further packs the primary packaging for retail purposes are examples of pharmaceutical packages. Hereinafter, pharmaceutical packages may be simply referred to as packages.

Referring to FIG. 1, the assisting device 1 is configured with a camera 2, a communication I/F unit 3, an operation input unit 4, a screen display unit 5, a storing unit 6, and an arithmetic processing unit 7.

The camera 2 is, for example, a color camera or a black-and-white camera each including a Charge-Coupled Device (CCD) image sensor or a complementary MOS (CMOS) image sensor having a pixel capacity of about several million pixels. The communication I/F unit 3 is configured with a data communication circuit and performs data communication with various external devices in a wireless or wired manner. The operation input unit 4 is configured with devices such as a keyboard and a mouse, and detects an operation by an expert who creates authenticity determination support information using the assisting device 1, and outputs it to the arithmetic processing unit 7. The screen display unit 5 is configured with a device such as a liquid crystal display (LCD), and displays on a screen various types of information in accordance with an instruction from the arithmetic processing unit 7.

The storing unit 6 is configured with one or a plurality of storage devices such as a hard disk and a memory, and stores therein processing information and a program 61 necessary for various types of processing by the arithmetic processing unit 7. The program 61 is a program that is read and executed by the arithmetic processing unit 7 to realize various processing units, and is loaded in advance from an external device or a storage medium that is not illustrated via a data input/output function of the communication I/F unit 3 or the like and stored in the storing unit 6. Main processing information stored in the storing unit 6 includes an authorized drug name list 62, an authentic drug image list 63, and an authenticity determination support information database 64.

The authorized drug name list 62 is a list of drug names authorized as pharmaceutical products by the national government agencies. The authorized drug name list 62 lists the authentic pharmaceutical names of all authorized pharmaceutical products.

The authentic drug image list 63 is a list of images obtained by capturing authentic (genuine) packages. The authentic drug image list 63 lists images of the front faces of all authentic (authentic) packages taken from right in front. Generally, the name of a pharmaceutical product is written in large letters on the front face of the package. The authentic drug image list 63 is a list of images of faces of packages in each of which the name of the pharmaceutical product is described in such large letters, captured from right in front. Further, in the authentic drug image list 63, an image obtained by capturing an authentic package is associated with the name of the pharmaceutical product described on the package. Therefore, by searching the authentic drug image list 63 with the pharmaceutical name as a keyword, it is possible to acquire an image obtained by capturing the front face of the authentic package on which the pharmaceutical name is described, from right in front.

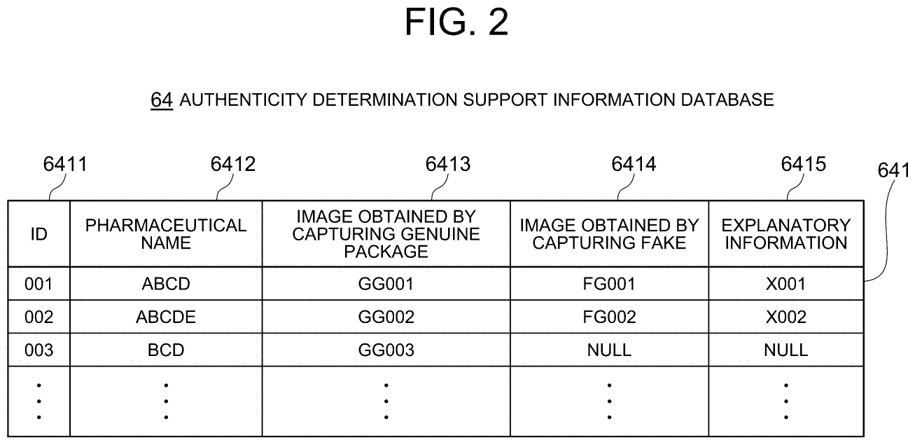

The authenticity determination support information database 64 is a database in which information (authenticity determination support information) for supporting the authenticity determination on a package by a user is accumulated. FIG. 2 is a diagram illustrating an example of information stored in the authenticity determination support information database. The authenticity determination support information database 64 of this example is configured with a plurality of pieces of authenticity determination support information 641. The individual authenticity determination support information 641 is configured with an ID 6411, pharmaceutical name 6412, an image 6413 obtained by capturing a genuine package, an image 6414 obtained by capturing a fake product, and explanatory information 6415.

In the entry of the ID 6411, an ID such as a number that uniquely identifies the authenticity determination support information is set. In the entry of the pharmaceutical name 6412, the pharmaceutical name of an authentic pharmaceutical name is set. In the entry of the image 6413 obtained by capturing a genuine package, an image obtained by capturing the front surface of the authentic package taken from right in front is set. In the entry of the image 6414 obtained by capturing a fake product, an image obtained by capturing the front surface of a fake product is set. In the entry of the explanatory information 6415, a figure and/or text explaining the key points for distinguishing between the image 6413 obtained by capturing an authentic package and the image 6414 obtained by capturing a fake one is set.

The arithmetic processing unit 7 includes one or a plurality of processors such as a Central Processing Unit (CPU) and peripheral circuits thereof, and reads the program 61 from the storing unit 6 and executes it, thereby making the hardware and the program 61 to cooperate to realize various processing units. The main processing units realized by the arithmetic processing unit 7 are an object image acquiring unit 71, a frontalized image generating unit 72, a pharmaceutical name recognizing unit 73, an authentic pharmaceutical name retrieving unit 74, an authentic product image retrieving unit 75, a difference detecting unit 76, and an editing unit 77.

The object image acquiring unit 71 is configured to acquire an image obtained by capturing a surface showing the pharmaceutical name of the package, from the camera 2 or from an external device via the communication I/F unit 3, as an object image.

The frontalized image generating unit 72 is configured to extract a region of the surface showing the pharmaceutical name from the object image acquired by the object image acquiring unit 71, and generate a frontalized image by performing trapezoidal correction on the extracted region of the surface.

The pharmaceutical name recognizing unit 73 is configured to recognize the pharmaceutical name by performing character recognition from the frontalized image generated by the frontalized image generating unit 72.

The authentic pharmaceutical name retrieving unit 74 is configured to retrieve a similar pharmaceutical name from the authorized drug name list 62 as an authentic pharmaceutical name by using the pharmaceutical name recognized by the pharmaceutical name recognizing unit 73.

The authentic product image retrieving unit 75 is configured to search the authentic drug image list 63 by using the authentic pharmaceutical name retrieved by the authentic pharmaceutical name retrieving unit 74 as a keyword to thereby acquire an image (authentic product image) obtained by capturing a surface showing the pharmaceutical name of the authentic package. The present example uses the authentic drug image list 63 as image information in which the authentic pharmaceutical name and the authentic product image obtained by capturing the surface showing the pharmaceutical name of the authentic pharmaceutical package are associated with each other, by performing image retrieval using the retrieved authentic pharmaceutical name as a keyword. However, the image information in which the authentic pharmaceutical name and the authentic product image obtained by capturing the surface showing the pharmaceutical name of the authentic pharmaceutical package are associated with each other is not limited to the authentic drug image list 63. For example, such image information may be a drug sales EC site or the like used in a second example embodiment which will be described later.

The difference detecting unit 76 is configured to detect a region with a large difference between the frontalized image generated by the frontalized image generating unit 72 and the authentic product image acquired by the authentic product image retrieving unit 75. The difference detecting unit 76 outputs, as a detection result, a frontalized image in which a region having a large difference from the authentic product image is marked with a rectangle shape or the like, for example,.

The editing unit 77 is configured to edit the authenticity determination support information by interactive processing with an expert. For example, the editing unit 77 displays, on the screen display unit 5, the object image acquired by the object image acquiring unit 71, the frontalized image generated by the frontalized image generating unit 72, the recognition result of the pharmaceutical name recognizing unit 73, the retrieval result of the authentic pharmaceutical name retrieving unit 74, the retrieval result of the authentic product image retrieving unit 75, and the detection result of the difference detecting unit 76, as editing materials, and allows an expert to check it and select or correct necessary editing materials by an input from the operation input unit 4. In addition, the editing unit 77 edits the authenticity determination support information using the editing materials selected or corrected by the expert and the text input from the operation input unit 4, and stores it into the authenticity determination support information database 64.

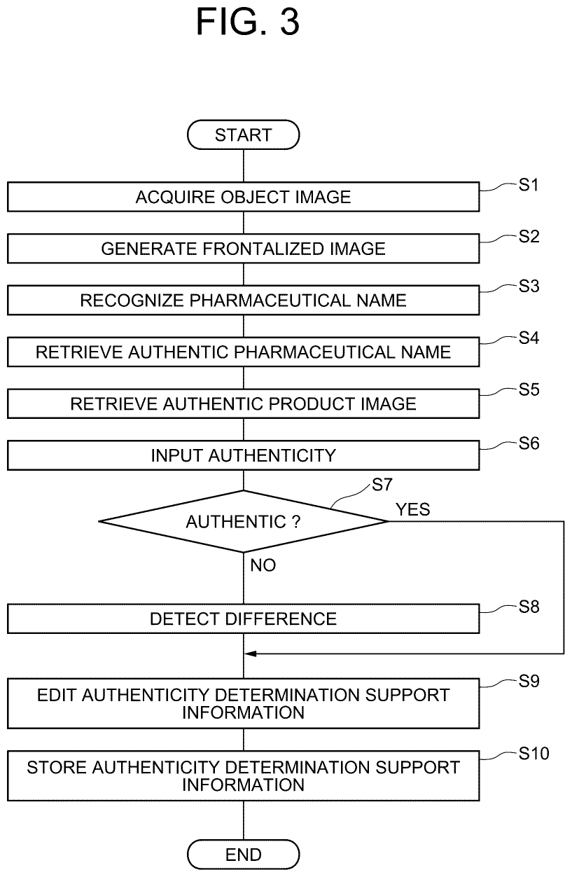

FIG. 3 is a flowchart illustrating an example of operation of the assisting device 1. Hereinafter, the operation of the assisting device 1 will be described with reference to FIG. 3.

First, the object image acquiring unit 71 of the assisting device 1 acquires, from the camera 2 or an external device via the communication I/F unit 3, an image obtained by capturing a surface showing the pharmaceutical name of the package as an object image (step S1). The acquired object image is displayed on the screen display unit 5 via the editing unit 77. This allows the expert to check the content of the object image. When the expert inputs OK for the displayed object image from the operation input unit 4, or when a certain time elapses without any input, the assisting device 1 continues processing as if the object image is accepted. On the other hand, when the expert inputs NG from the operation input unit 4 within a certain period of time due to a reason such as no package being shown on the object image, the assisting device 1 interrupts the processing of FIG. 3 at this point of time.

Next, the frontalized image generating unit 72 extracts a region of the surface showing the pharmaceutical name from the object image acquired by the object image acquiring unit 71, and then generates a frontalized image by performing trapezoidal correction on the extracted region of the surface (step S2). The generated frontalized image is displayed on the screen display unit 5 via the editing unit 77. This allows the expert to check the content of the frontalized image. When the expert inputs OK for the displayed frontalized image from the operation input unit 4, or when a certain period of time elapses without any input, the assisting device 1 continues processing as if the frontalized image is accepted. On the other hand, when the expert inputs NG from the operation input unit 4 within a certain period of time due to a reason such as the image not being frontalized clearly, the assisting device 1 interrupts the processing of FIG. 3 at this point of time.

Next, the pharmaceutical name recognizing unit 73 recognizes the pharmaceutical name by performing character recognition from the frontalized image generated by the frontalized image generating unit 72 (step S3). The recognized pharmaceutical name is displayed on the screen display unit 5 via the editing unit 77. This allows the expert to check the recognized pharmaceutical name. When the expert inputs OK for the recognized pharmaceutical name from the operation input unit 4, or when a certain period of time elapses without any input, the assisting device 1 continues processing as if the recognized pharmaceutical name is accepted. On the other hand, when the expert corrects the recognized pharmaceutical name from the operation input unit 4 within a certain period of time because the recognized pharmaceutical name is incorrect, the assisting device 1 adopts the corrected pharmaceutical name as the recognized pharmaceutical name and continues processing.

Next, the authentic pharmaceutical name retrieving unit 74 retrieves similar pharmaceutical names from the authorized pharmaceutical names list 74 as the authentic pharmaceutical name, by using the pharmaceutical name recognized by the pharmaceutical name recognizing unit 73 (step S4). The retrieval results of the authentic pharmaceutical name are displayed on the screen display unit 5 via the editing unit 77. This allows the experts to check the retrieval results of the authentic pharmaceutical name. When the expert inputs OK from the operation input unit 4 for a retrieved authentic pharmaceutical name with the highest degree of similarity, or when a certain period of time elapses without any input, the assisting device 1 continues processing as if the retrieved authentic pharmaceutical name with the highest degree of similarity is accepted. On the other hand, when the expert corrects the authentic pharmaceutical name from the operation input unit 4 within a certain period of time because the authentic pharmaceutical name with the highest degree of similarity is incorrect, the assisting device 1 adopts the corrected authentic pharmaceutical name with the highest degree of similarity as the authentic pharmaceutical name with the highest degree of similarity and continues processing.

Next, the authentic product image retrieving unit 75 searches the authentic drug image list 63 using the authentic pharmaceutical name with the highest degree of similarity retrieved by the authentic pharmaceutical name retrieving unit 74 as a keyword, and retrieves an image (authentic product image) obtained by capturing the surface showing the pharmaceutical name of the authentic package (step S5). The retrieval result of the authentic product image is displayed on the screen display unit 5 via the editing unit 77. As a result, the expert can check the retrieval result of the authentic product image. When the expert inputs OK for the retrieved authentic product image with the highest degree of similarity from the operation input unit 4, or when a certain period of time elapses without any input, the assisting device 1 continues processing as if the retrieved authentic product image with the highest degree of similarity is accepted. On the other hand, when the expert selects another authentic product image retrieved within a certain period of time from the operation input unit 4 because the authentic product image with the highest degree of similarity is incorrect, the assisting device 1 adopts the selected authentic product image as the authentic product image with the highest degree of similarity, and continues processing.

Next, the editing unit 77 receives a view on whether the object image displayed on the screen display unit 5 (the image acquired in step S1) is genuine (authentic) or fake from the operation input unit 4 (step S6). The expert determines whether the package pertaining to the object image is genuine or fake on the basis of the object image displayed on the screen display unit 5, the frontalized image, the authentic pharmaceutical name, the normalized image, and the expertise of authenticity determination, and inputs the determination result from the operation input unit 4.

Then, when a determination result that it is fake is input (NO in step S7), the assisting device 1 proceeds to the process of step S8. Alternatively, when a determination result that it is genuine is input (YES in step S7), the assisting device 1 skips step S8 and proceeds to the process of step S9.

In step S8, the difference detecting unit 76 detects a region with a large difference between the frontalized image generated by the frontalized image generating unit 72 and the authentic product image acquired by the authentic product image retrieving unit 75 (step S6).

The detection result of the difference detecting unit 76 is displayed on the screen display unit 5 via the editing unit 77. As a result, the expert can check presence/absence of a region with a large difference from the authentic product image and the location thereof on the frontalized image.

Further, the expert can modify the detection result of the difference detecting unit 76 by means of an input operation from the operation input unit 4. For example, when the expert finds that there is a large difference between the two in another part where it is determined that there is no large difference between the two in the detection result of the difference detecting unit 76, the expert adds a marking such as a rectangular shape to the found part. On the other hand, when the expert finds that there is no significant difference between the two parts determined to have a large difference in the detection result of the difference detecting unit 76, the expert deletes the marking of the found part. When, for example, confirmation or completion of modification is input from the operation input unit 4 by the expert, the assisting device 1 proceeds to the process of step S9.

In step S9, the editing unit 77 edits the authenticity determination support information by using the editing material selected or modified by the expert and the authenticity determination result and the text input from the operation input unit 4, and stores the information into the authenticity determination support information database 64 (step S10).

Next, a specific example of the authenticity determination support information created using the assisting device 1 will be described.

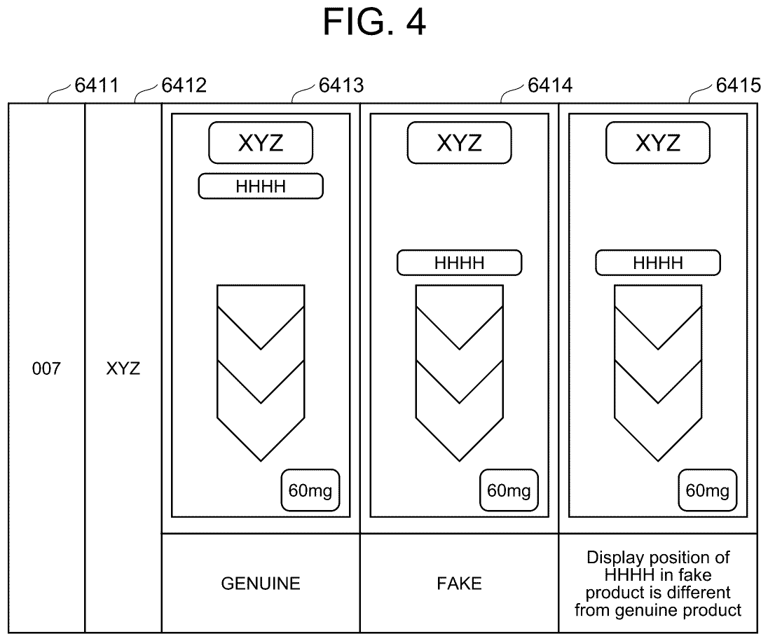

FIG. 4 is a schematic diagram illustrating an example of authenticity determination support information created using the assisting device 1. This example is an example where an object image is an image of a fake package. In the authenticity determination support information of this example, “007” is set in the entry of the ID 6411, and “XYZ” is set in the entry of the drug name 6412. “007” set in the entry of the ID 6411 is, for example, a number automatically adopted by the editing unit 77, or an ID attached to the input object image. “XYZ” set in the entry of the pharmaceutical name 6412 is the authentic pharmaceutical name retrieved by the authentic pharmaceutical name retrieving unit 74 and approved by the expert. In addition, the authentic product image retrieved by the authentic product image retrieving unit 75 and approved by the expert is set in the entry of the image 6413 obtained by capturing a genuine package. Further, the frontalized image generated by the frontalized image generating unit 72 is set in the entry of the image 6414 obtained by capturing a fake product. Further, in the entry of the explanatory information 6415, the detection result detected by the difference detecting unit 76 (frontalized image in which a region with a large difference from the authentic product image is marked with a rectangular shape or the like) and the text input by the expert “Display position of HHHH in fake product is different from genuine product” are set. In the image of the authentic product set in the entry 6413 and the fake image set in the entry 6414, the size of the character “60” and the shape of the figure are slightly different between the authentic product and the fake product. However, since the difference between them is not a large difference, it is not marked in the frontalized image of the fake product set in the entry 6415, nor is it explained in the text entered by the expert. By not mentioning the difference that is difficult to understand for general users intentionally, important checkpoints of authenticity determination are highlighted.

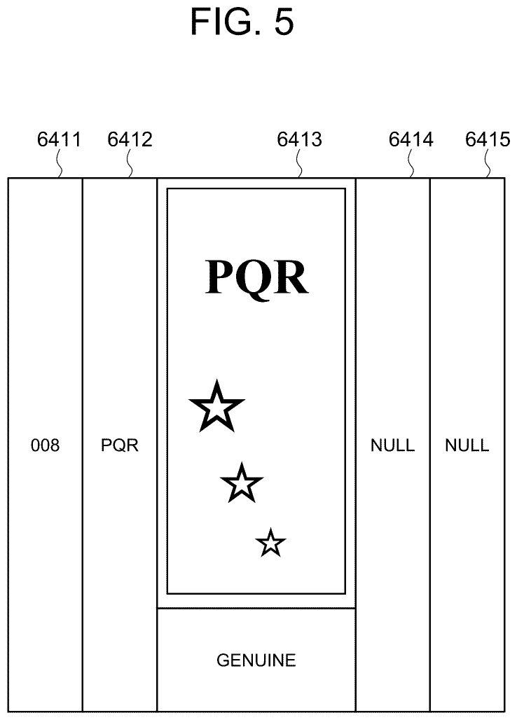

FIG. 5 is a schematic diagram illustrating another example of authenticity determination support information created using the assisting device 1. This example is an example where an object image is an image of a genuine package. Therefore, in the authenticity determination support information, the ID, the authentic pharmaceutical name, and the authentic product image are set in the entries of the ID 6411, the pharmaceutical name 6412, and the image 6413 obtained by capturing a genuine package, and NULL is set in the entries of the image 6414 obtained by capturing a fake product and the explanatory information 6415.

As described above, the assisting device 1 includes the frontalized image generating unit 72 that extracts the above-described region of a surface from the object image obtained by capturing the surface showing the pharmaceutical name of a pharmaceutical package and generates a frontalized image by performing trapezoidal correction, and the pharmaceutical name recognizing unit 73 that recognizes the pharmaceutical name by performing character recognition from the generated frontalized image. Therefore, since the expert only needs to check the recognized pharmaceutical name, there is no need to visually read the pharmaceutical name from the object image and input it manually from the operation input unit 4. Further, if the shape deformation of the object image is large, it may be difficult to apply character recognition, or the possibility of misrecognition may increase. However, since character recognition is performed after the trapezoidal correction is performed and the frontalized image is generated by the frontalized image generating unit 72, there is no such problem, and character recognition can be performed with high accuracy.

In addition, the assisting device 1 includes the authentic pharmaceutical name retrieving unit 74 that retrieves a similar pharmaceutical name from the authorized pharmaceutical name list 62 by using the recognized pharmaceutical name, as the authentic pharmaceutical name.

Therefore, the expert can save time and efforts for searching for whether or not it is an authorized pharmaceutical product by himself/herself.

In addition, the assisting device 1 includes the authentic product image retrieving unit 75 that retrieves an authentic product image obtained by capturing a surface showing the pharmaceutical name of an authentic pharmaceutical package by image retrieval using the authentic pharmaceutical name as a keyword. As a result, an expert can save the time and efforts to perform image retrieval and acquire the authentic image by himself/herself.

As described above, since the expert does not need to search for whether or not it is an authorized pharmaceutical product or search for an authentic product image by himself/herself, the expert can spend enough time determining whether the object image is an image of a genuine package or a fake one.

Further, the assisting device 1 includes the difference detecting unit 76 that detects a region with a large difference between the frontalized image and the authentic product image. Therefore, the expert only needs to check the detected difference, so that the expert can save the time and efforts to find and draw the difference between the two. In addition, variations in the difference found by respective experts can be suppressed.

As described above, the time and cost in creating the authenticity determination support information can be reduced by using the assisting device 1.

Next, preferred examples of main components will be described.

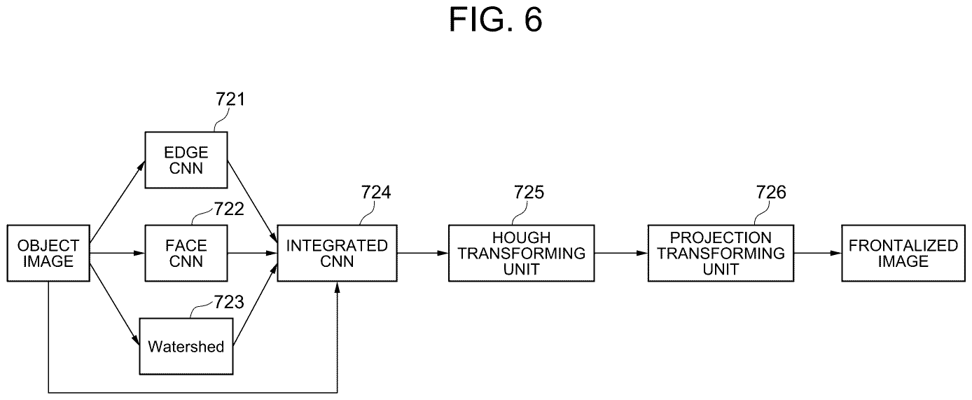

FIG. 6 is a block diagram illustrating an example configuration of the frontalized image generating unit 72. The frontalized image generating unit 72 in this example is configured with an edge Convolutional Neural Network (CNN) 721, a face CNN 722, a watershed 723, an integrated CNN 724, a Hough transforming unit 725, and a projection transforming unit 726. The frontalized image generating unit 72 of this example inputs an object image into the edge CNN 721, the plane CNN 722, the watershed 723, and the integrated CNN 724.

The edge CNN 721 detects an edge of a package shown in the input object image, and outputs, for example, an object image in which the edge is shown in red. The face CNN 722 detects a surface of the package shown in the input object image, and outputs, for example, an object image in which the surface is shown in red. The watershed 723 separates an object such as a package from the background shown in the input object image, and outputs, for example, an object image in which the background is shown in yellow. The integrated CNN 724 outputs an object image in which the surface of the package shown in the object image is almost completely shown in red, from the object image, the output image of the edge CNN 721, the output image of the face CNN 722, and the output image of the watershed 723. The Hough transforming unit 725 inputs therein the output image of the integrated CNN 724, detects the outer contour of the surface of the package, and outputs an object image in which, for example, the outer contour is shown in red. The projection transforming unit 726 inputs therein the output image of the Hough transforming unit 725, applies trapezoidal correction to the shape of the surface of the package specified by the outer contour, and generates a frontalized image.

An example configuration of the frontal image generating unit 72 has been described above. However, the frontalized image generating unit 72 is not limited to the above configuration example. It is possible to apply a well-known technique for performing trapezoidal correction on the surface shown in the image, which is originally in a rectangular shape but distorted into a trapezoidal shape.

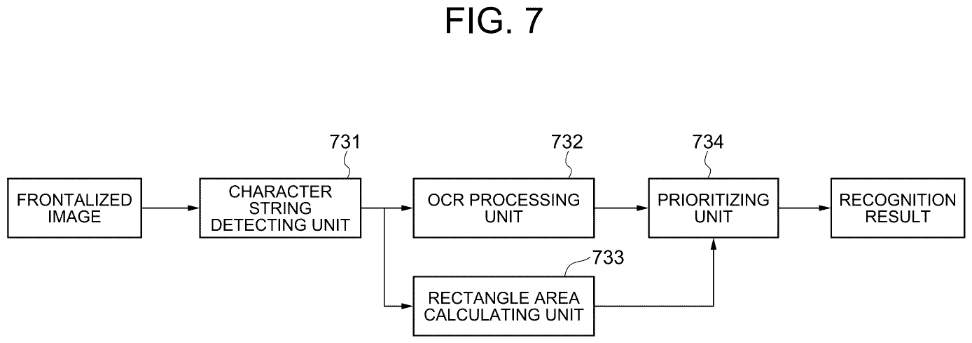

FIG. 7 is a block diagram illustrating an example configuration of the pharmaceutical name recognizing unit 73. The pharmaceutical name recognizing unit 73 of this example is configured with a character string detecting unit 731, an optical character recognition (OCR) processing unit 732, a rectangle area calculating unit 733, and a prioritizing unit 734.

The character string detecting unit 731 inputs therein a frontalized image, and detects all the circumscribed rectangle shapes of the character strings shown in the frontalized image. For example, the character string detecting unit 731 detects a rectangle region in which shapes (characters) of similar size are arranged at certain intervals as one character string. The OCR processing unit 732 performs OCR processing on each rectangle region detected by the character string detecting unit 731 to perform character recognition on the inside of the rectangle region, and outputs the recognition result. On the other hand, the rectangle area calculating unit 733 calculates and outputs the area of the rectangle region detected by the character string detecting unit 731. The prioritizing unit 734 outputs a recognition result in which the recognition results output from the OCR processing unit 732 are prioritized in such a manner that the larger the area of the rectangle region output from the rectangle area calculation unit 733, the higher the order.

The pharmaceutical name recognizing unit 73 of this example focuses on the fact that, although various character strings may exist on the package apart from the pharmaceutical name, the pharmaceutical name is most notated. In the verification by the inventor, the probability of the highest ranking recognition result being the pharmaceutical name was almost 100%. This effect is also related to the function of the frontalized image generating unit 72. That is to say, since the size of the character string shown in the image obtained by capturing the package from an angle is distorted, the pharmaceutical name may be the same size as or smaller than that of other character strings depending on the posture taken. On the other hand, there is no such negative effect in the frontalized image.

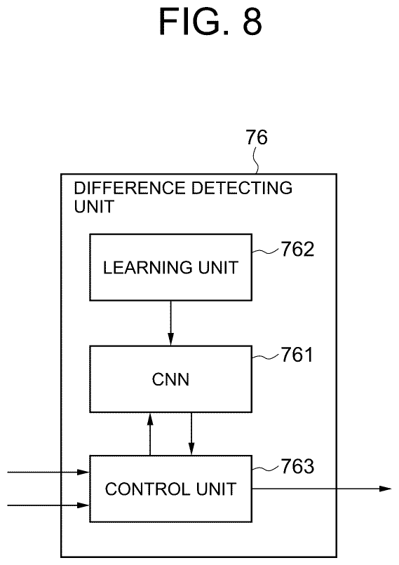

FIG. 8 is a block diagram illustrating an example configuration of the difference detecting unit 76. The difference detecting unit 76 of this example includes a CNN 761, a learning unit 762, and a control unit 763.

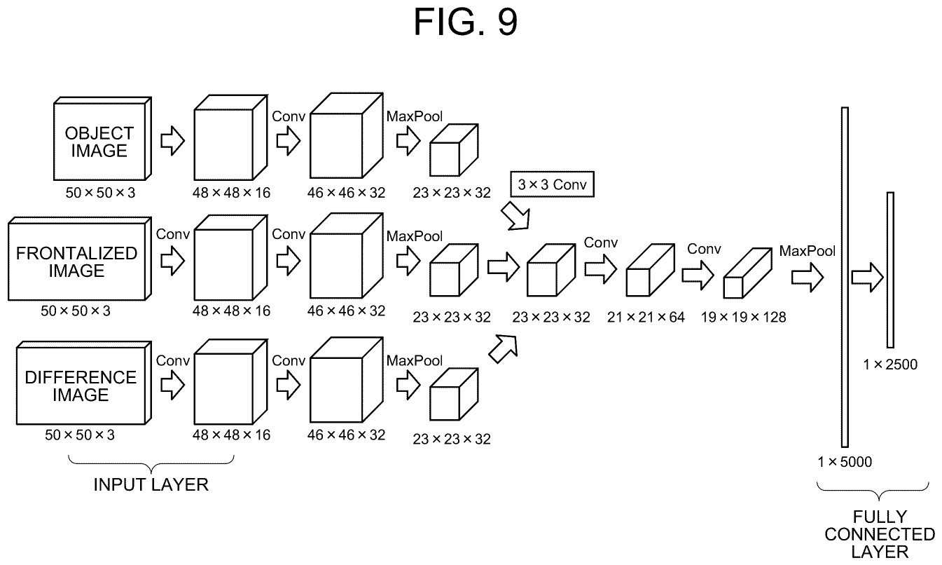

The CNN 761 is a trained model in which machine learning has been performed for estimating a region with a large difference between a frontalized image and an authentic product image by inputting therein a frontalized image generated by the frontalized image generating unit 72, an authentic product image retrieved by the authentic product image retrieving unit 75, and a difference image between these two images (frontalized image and authentic product image). FIG. 9 illustrates an example of the CNN 761. However, the CNN 761 is not limited to have the configuration illustrated in FIG. 9.

The learning unit 762 is a means for performing machine learning of the CNN 761 so as to estimate a region having a large difference using various pairs of frontalized images and authentic product images as training data. As methods of teaching a region having a large difference between a pair of a frontalized image and an authentic product image, there are methods such as enclosing a region with a large difference with a rectangle, filling the region, and the like.

The control unit 763 inputs therein the frontalized image generated by the frontalized image generating unit 72 and the authentic product image retrieved by the authentic product image retrieving unit 75, and generates a difference image between the two images (frontalized image and authentic product image). Further, the control unit 763 is configured to acquire a region with a large difference between the frontalized image and the authentic product image from the CNN 761 by inputting the input frontalized image and authentic product image and the generated difference image into the trained CNN 761.

The configuration and operation of the assisting device 1 have been described above.

Next, a system for supporting a user in determining authenticity of a package by using the authenticity determination support information created using the assisting device 1 will be described.

### Second Example Embodiment

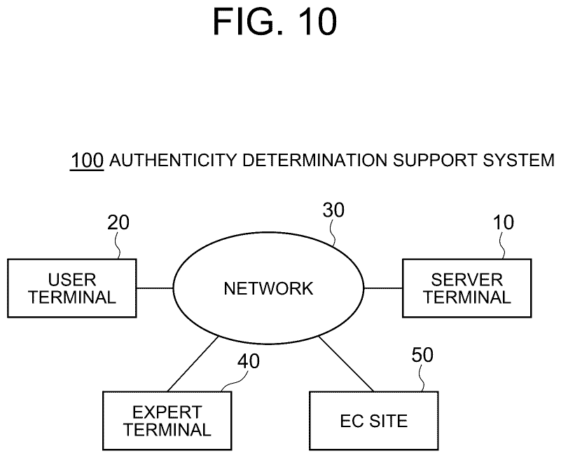

FIG. 10 is a block diagram of an authenticity determination support system 100 according to a second example embodiment of the present invention. In the authenticity determination support system 100, a package is an object of authenticity determination. Referring to FIG. 10, the authenticity determination support system 100 is configured with a server terminal 10 and a user terminal 20, the two of which are communicably connected to each other over a network 30 such as the Internet. The user terminal 20 is a terminal used by a user who makes an authenticity determination of a package. The user terminal 20 may be an information processing device such as a smartphone, a personal digital assistant, or a personal computer having a camera function, a communication function, and a display function. On the other hand, the server terminal 10 is a terminal that provides, to the user terminal 20, information that supports authenticity determination of a package in accordance with a request from the user terminal 20. The server terminal 10 may be an information processing device such as a personal computer having a communication function, an arithmetic function, and a storage function.

For example, when the user of the user terminal 20 desires to find out whether the package he/she is about to purchase is genuine or fake, the camera function of the user terminal 20 is used to acquire an image obtained by capturing the appearance of the package. Then, the user terminal 20 sends a support request including the acquired image to the server terminal 10 over the network 30. Then, the user terminal 20 waits for a response from the server terminal 10.

The server terminal 10 stores therein the authenticity determination support information created by an expert using the assisting device 1 described above in advance. In response to receiving the support request from the user terminal 20 over the network 30, the server terminal 10 matches the image obtained by capturing a package included in the support request against images in the authenticity determination support information stored in advance (including both an image obtained by capturing a genuine package and an image obtained by capturing a fake product). Then, as a result of matching, in the case where authenticity determination support information including an image matching the image obtained by capturing the package included in the support request (including at least one of an image obtained by capturing a genuine package and an image obtained by capturing a fake) is detected, the server terminal 10 sends the detected authenticity determination support information to the user terminal 20.

When the authenticity determination support information is sent from the server terminal 10, the user terminal 20 receives it and displays it on the screen. Consequently, the user can check not only the image obtained by capturing a genuine package but also the image obtained by capturing a fake, with respect to the package that is an authenticity determination object. Therefore, the user can determine the degree of similarity between the image obtained by capturing the package that is an authenticity determination object and the images obtained by capturing genuine and fake objects from a more multifaceted perspective than the case where it is necessary to determine whether the package that is an authenticity determination object is genuine or fake according to only an image obtained by capturing a genuine package, so that the authenticity determination can be performed easily and with high accuracy. In addition, the text and/or figures explaining the essential points for distinguishing an image obtained by capturing a genuine package from an image obtained by capturing a fake are displayed, so that the user can perform authenticity determination more easily with higher accuracy.

Further, in the authenticity determination support system 100 illustrated in FIG. 10, an expert terminal 40 and an electronic commerce (EC) site 50 are connected to the network 30. The expert terminal 40 is a terminal used by an expert who makes an authenticity determination on the package. The expert terminal 40 may be an information processing device such as a smartphone, a personal digital assistant, and a personal computer having a camera function, a communication function, and a display function. To be specific, the expert terminal 40 may be the assisting device 1 according to the first example embodiment. In addition, the EC site 50 is a website that sells packages. The EC site 50 discloses images of the packages to be sold in association with the product names.

Next, the configuration and operation of the server terminal 10 will be described in detail.

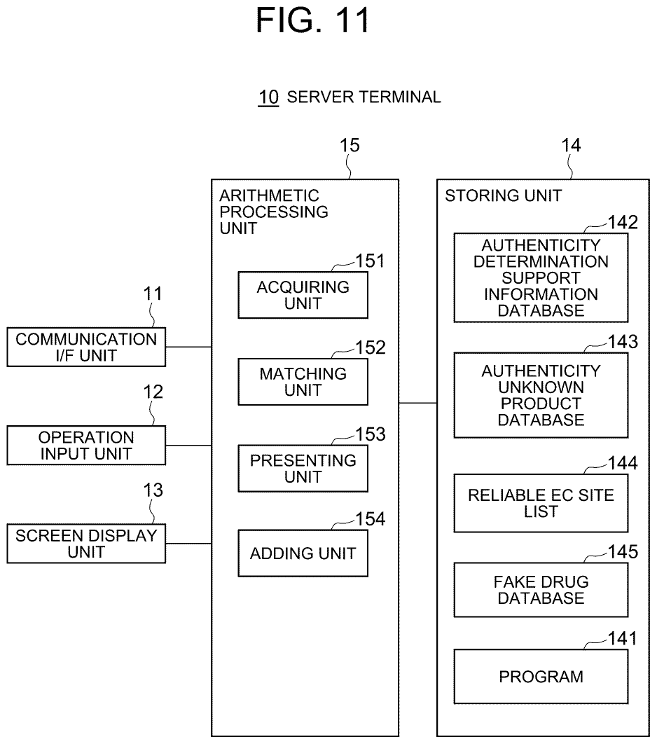

FIG. 11 is a block diagram illustrating an example of the server terminal 10. Referring to FIG. 11, the server terminal 10 is configured with a communication I/F unit 11, an operation input unit 12, a screen display unit 13, a storing unit 14, and an arithmetic processing unit 15.

The communication I/F unit 11 is configured with a data communication circuit, and performs data communication with external devices such as the user terminal 20, the expert terminal 40, and the EC site 50 over the network 30 in a wireless or wired manner. The operation input unit 12 is configured with devices such as a keyboard and a mouse, and detects an operation by an operator and outputs it to the arithmetic processing unit 15. The screen display unit 13 is configured with a device such as an LCD, and displays, on a screen, various types of information in accordance with an instruction from the arithmetic processing unit 15.

The storing unit 14 is configured with one or a plurality of storage devices such as a hard disk and a memory, and stores therein processing information and a program 141 necessary for various types of processing by the arithmetic processing unit 15. The program 141 is a program that realizes various processing units by the arithmetic processing unit 15 reading into and executing, and is loaded in advance from an external device or a storage medium, not illustrated, via the data input/output function of the communication I/F unit 11 and the like and stored in the storing unit 14. Main processing information stored in the storing unit 14 includes an authenticity determination support information database 142, an authenticity unknown product database 143, a reliable EC site list 144, and a fake drug database 145.

The authenticity determination support information database 142 is a database constructed using the aforementioned authenticity determination support information database 64 prepared in advance by an expert using the assisting device 1. The configuration of the individual authenticity determination support information 641 stored in the authenticity support information database 142 is the same as the configuration illustrated in FIG. 2. In the example of FIG. 11, the authenticity determination support information database 142 is locally connected to the server terminal 10, but may be remotely connected to the server terminal 10.

The authenticity unknown product database 143 temporarily stores therein an image obtained by capturing an authenticity unknown package. In the example of FIG. 11, the authenticity unknown product database 143 is connected to the server terminal 10 locally, but it may be connected remotely. In the case where it is confirmed by the expert that an image stored in the authenticity unknown product database 143 is an image obtained by capturing a genuine package or an image obtained by capturing a fake package, new authenticity determination support information including such image is registered in the authenticity determination support information database 142, and the original image is deleted from the authenticity unknown product database 143.

In the reliable EC site list 144, the EC sites 50 that sell packages and that have been confirmed to be reliable in the prior investigation are listed. The EC sites on the list have disclosed images of the packages to be sold in association with the product names. Therefore, by searching the Internet for an image by using the product name of a pharmaceutical product as a keyword, it is possible to acquire images obtained by capturing a genuine package provided by those websites. Some of the images obtained by capturing the genuine package provided by those websites may match or do not match an image obtained by capturing a genuine package registered in the authenticity determination support information database 142.

The fake drug database 145 stores therein an image obtained by capturing a fake package in advance. In the example of FIG. 11, the fake drug database 145 is locally connected to the server terminal 10, but it may be remotely connected. Some of the images obtained by capturing fake packages stored in the fake drug database 145 may match or not match an image obtained by capturing a fake package registered in the authenticity determination support information database 142.

The arithmetic processing unit 15 includes one or a plurality of processors such as a CPU and peripheral circuits thereof, and reads the program 141 from the storing unit 14 and executes it, thereby making the hardware and the program 141 to cooperate and realize various processing units. The main processing units realized by the arithmetic processing unit 15 include an acquiring unit 151, a matching unit 152, a presenting unit 153, and an adding unit 154.

The acquiring unit 151 is configured to communicate with the user terminal 20 over the network 30 via the communication I/F unit 11, and acquire a support request including an image obtained by capturing a package that is an authenticity determination object (hereinafter referred to as a determination object image) from the user terminal 20.

The matching unit 152 is configured to match the determination object image included in the support request received from the acquiring unit 151 against the image 6413 obtained by capturing a genuine package and the image 6414 obtained by capturing a fake package in every authenticity determination support information 641 stored in the authenticity determination support information database 142. For example, a subject identification engine may be used for image matching. The matching unit 152 detects the authenticity determination support information 641 in which at least one of the image 6413 obtained by capturing a genuine package or the image 6414 obtained by capturing a fake package matches the determination object image. The matching unit 152 transmits the matching result including the ID 6411 of the detected authenticity determination support information 641 to the presenting unit 153. Further, in the case where no authenticity determination support information 641 as described above is detected, the matching unit 152 transmits the matching result not including the ID 6411 of the authenticity determination support information 641 at all, to the presenting unit 153. Moreover, the matching unit 152 assigns an authenticity unknown product ID to the determination object image in which no authenticity determination support information 641 has been detected, and stores it in the authenticity product database 143.

The presenting unit 153 is configured to present information supporting the authenticity determination of the determination object to the user of the user terminal 20. The presenting unit 153 checks whether or not the ID 6411 is included in the matching result transmitted from the matching unit 152, and when it is included, creates support information based on the authenticity determination support information database 142, and when it is not included, creates support information based on the EC site 50 and the fake drug database 145.

In the case of creating the support information based on the authenticity determination support information database 142, the presenting unit 153 reads the authenticity determination support information 641 having the ID 6411 from the authenticity determination support information database 142 using the ID 6411 included in the matching result as a key, and creates the support information based on the readout authenticity determination support information 641. For example, the presenting unit 153 uses the authenticity determination support information 641 as the support information as it is.

In the case of creating the support information based on the EC site, the presenting unit 153 first extracts the product name from the determination object image. Then, the presenting unit 153 searches the Internet for an image using the extracted product name as a keyword.

Through the search for images, images obtained by capturing a genuine package that is sold under the extracted product name are obtained. Then, the presenting unit 153 extracts only images provided by the EC sites described in the reliable EC site list 144 among the images of the packages obtained through the search. Then, the presenting unit 153 matches the extracted images against the determination object image, and detects an image obtained by capturing a genuine package that matches the determination object image. Then, the presenting unit 153 creates support information including the detected image as an example of an image obtained by capturing a genuine package. When there is no image obtained by capturing a genuine package that matches the determination object image, the presenting unit 153 creates support information indicating the fact.

In the case of creating support information based on the fake medicine database 145, the presenting unit 153 first matches an image obtained by capturing a fake package stored in the fake drug database 145 against the determination object image to thereby detect an image obtained by capturing a fake package that matches the determination object image. Then, the presenting unit 153 creates support information including the detected image obtained by capturing a fake package as an example. When no image obtained by capturing a fake package matching the determination object image is detected, the presenting unit 153 creates support information indicating the fact.

The presenting unit 153 transmits the support information created as described above to the user terminal 20 requesting support over the network 30 via the communication I/F unit 11, and causes the support information to be displayed on the screen of the user terminal 20. There are various possible forms in which the presenting unit 153 causes the support information to be displayed on the screen of the user terminal 20. For example, the presenting unit 153 may display the image obtained by capturing a genuine package included in the support information, the image obtained by capturing a fake, and explanation information at the same time. Alternatively, the presenting unit 153 may allow the image obtained by capturing a genuine package included in the support information and the image obtained by capturing a fake package to be displayed alternately, and allow the explanation information to be displayed only when the user requests displaying of the explanation information.

The adding unit 154 is configured to collect authenticity determination support information related to a determination object image whose authenticity is unknown from experts and store it in the authenticity determination support information database 142 to thereby enhance the authenticity determination support information database 142. For example, when a determination object image assigned with an authenticity unknown product ID is newly stored in the authenticity unknown product database 143 by the matching unit 152, the adding unit 154 transmits the authenticity product information to the expert terminal 40. The adding unit 154 includes a determination object image that has been assigned with at least an authenticity unknown product ID in the authenticity unknown product information.

Moreover, when new authenticity determination support information is transmitted from the expert terminal 40, the adding unit 154 receives it and stores it into the authenticity determination support information database 142. Here, the format of the authenticity determination support information transmitted from the expert terminal 40 is the same as that of the authenticity determination support information 641 illustrated in FIG. 2 However, the authentication unknown product ID has been set by an expert.

That is to say, an expert determines whether the determination object image received from the server terminal 10 is an image obtained by capturing a genuine package or a fake package, using the assisting device 1 according to the first example embodiment and his/her own expertise. Further, in the case where the expert determines that the determination object image is an image obtained by capturing a genuine package, the expert creates the authenticity determination support information 641 be recording the authenticity unknown product ID in the entry of the ID 6411, recording the pharmaceutical name of the genuine package in the entry of the pharmaceutical name 6412, recording the determination object image or another image obtained by capturing a genuine package in the entry of the image 6413 obtained by capturing a genuine package, and setting NULL in the entries of the image 6414 obtained by capturing a fake and explanatory information 6415. However, in the case where the expert has acquired an image obtained by capturing a fake of a genuine package through any route, the image obtained by capturing the fake may be set in the entry of the image 6414, and text and/or figure explaining the essential points for distinguishing between the image 6414 obtained by capturing a genuine package and the image 6414 obtained by capturing a fake package may be recorded in the entry of the explanatory information 6415. In addition, when the expert determines that the determination object image is an image obtained by capturing a fake package, the expert creates the authenticity determination support information 641 by recording the authenticity unknown product ID in the entry of the ID 6411, recording the pharmaceutical name of a genuine package and an image of such package serving as the grounds for determining that the package is fake in the entries of the pharmaceutical name 6412 and the image 6413 obtained by capturing a genuine package, recording the determination object image in the entry of the image 6414 obtained by capturing a face package, and recording text and/or figures explaining the key points for distinguishing between the image 163 obtained by capturing a genuine package and the image 164 obtained by capturing a fake in the entry of the explanatory information 6415.

Then, the expert sends the authenticity determination support information 641 created as described above, from the expert terminal 40 to the server terminal 10 over the network 30. When the adding unit 154 receives the authenticity determination support information 641 via the communication I/F unit 11, the adding unit 154 rewrites the entry of the ID 6411 into an ID that can be uniquely identified in the authenticity determination support information database 142, and registers in the authenticity determination support information database 142. Then, the adding unit 154 deletes the determination object image having the authenticity unknown product ID before rewriting from the authenticity unknown product database 143.

Next, the operation of the server terminal 10 will be described.

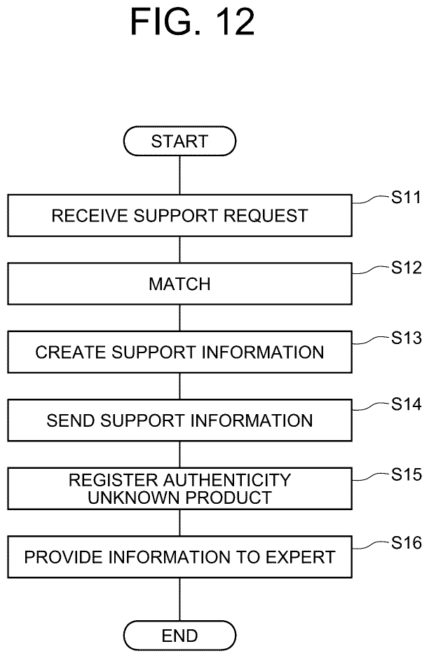

FIG. 12 is a flowchart illustrating an example of operation of the server terminal 10 when a support request is sent from the user terminal 20 to the server terminal 10. Referring to FIG. 12, the acquiring unit 151 of the server terminal 10 receives a support request sent from the user terminal 20 (step S11). Then, the matching unit 152 of the server terminal 10 matches the determination object image included in the support request against the image 6413 obtained by capturing a genuine package and the image 6414 obtained by capturing a fake in every authenticity determination support information 641 stored in the authenticity determination support information database 142 (step S12). In step S12, in the case where the authenticity determination support information 641 in which at least one of the image 6413 obtained by capturing a genuine package or the image 6414 obtained by capturing a fake matches the determination object image is detected, the matching unit 152 transmits the matching result including the ID 6411 of the detected authenticity determination support information 641 to the presenting unit 153. In the case where no authenticity determination support information 641 as described above is detected in step S12, the matching unit 152 transmits the matching result not including the ID 6411 of the authenticity determination support information 641 to the presenting unit 153.

Then, the presenting unit 153 creates information that supports authenticity determination of the determination object (step S13). In step S13, the presenting unit 153 checks whether or not the ID 6411 is included in the matching result, and when it is included, the presenting unit 153 creates support information based on the authenticity determination support information database 142 by means of the method as described above, and when it is not included, the presenting unit 153 creates support information based on the EC site 50 and the fake drug database 145 by means of the method as described above. Then, the presenting unit 153 sends the created support information to the user terminal 20 requesting support, over the network 30 via the communication I/F unit 11, and causes the support information to be displayed on the screen of the user terminal 20 (step S14).

In addition, the matching unit 152 assigns an authenticity unknown product ID to the determination object image in which no authenticity determination support information 641 is detected in the matching at step S2, and stores it into the authenticity unknown product database 143 (step S15). Further, when a new determination object image is stored in the authenticity unknown product database 143, the adding unit 154 sends authenticity unknown product information including the determination object image assigned with an authenticity unknown product ID, to the expert terminal 40 (step S16). When the expert terminal 40 receives the authenticity unknown product information including the determination object image sent from the server terminal 10, the expert of the expert terminal 40 is notified of the fact. The expert performs a process similar to the process described in FIG. 3 based on the received determination object image to thereby create the authenticity determination support information 641. Then, the expert sends the created authenticity determination support information 641 from the expert terminal 40 to the server terminal 10 via the communication I/F unit.

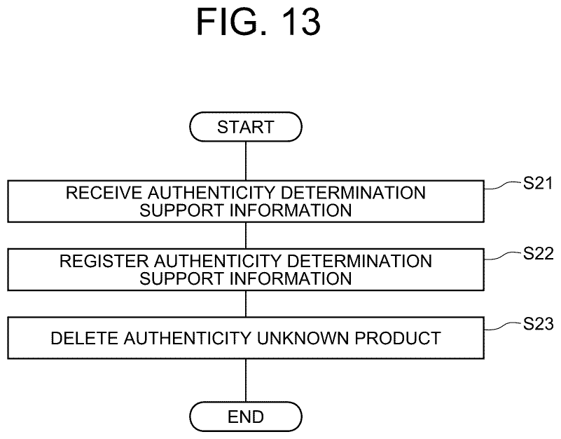

FIG. 13 is a flowchart illustrating an example of operation of the server terminal 10 when the authenticity determination support information is sent from the expert terminal 40 to the server terminal 10. Referring to FIG. 13, the adding unit 154 of the server terminal 10 receives the authenticity determination support information 641 sent from the expert terminal 40 (step S21). Then, the adding unit 154 rewrites the ID 6411 of the received authenticity determination support information from the authenticity unknown product ID to the ID of the authenticity determination support information, and registers it in the authenticity determination support information database 142 (step S22). Then, the adding unit 154 deletes the determination object image having the authenticity unknown product ID before rewriting from the authenticity unknown product database 143 (step S23).

In this manner, the server terminal 10 matches the determination object image included in the support request sent from the user terminal 20 against the image 6413 obtained by capturing a genuine package and the image 6414 obtained by capturing a fake in every authenticity determination support information 641 stored in the authenticity determination support information database 142. Then, in the case where the authenticity determination support information 641 in which at least one of the image 6413 obtained by capturing a genuine package and the image 6414 obtained by capturing a fake matches the determination object image is detected, the server terminal 10 creates support information including the image obtained by capturing a genuine package, the image obtained by capturing a fake, and text and/or an image explaining the key points for distinguishing between the two, on the basis of the detected authenticity determination support information 641, and causes it to be displayed on the screen of the user terminal 20. Consequently, the user can easily and accurately perform the authenticity determination.

In the case where the authenticity determination support information 641 that matches the determination object image is not detected, the server terminal 10 creates support information based on the result of searching for an image matching the determination object image from the reliable EC site 50 and the fake drug database 145, and causes it to be displayed on the screen of the user terminal 20. Consequently, it is possible for the user to make an authenticity determination with a certain accuracy.

In addition, in the case where the authenticity determination support information 641 that matches the determination object image is not detected, the server terminal 10 stores the determination object image in the authenticity unknown item database 143, and sends the determination object image to the expert terminal 40. Then, when the expert determines the authenticity of the determination object image and sends the authenticity determination support information created by the expert based on the result from the expert terminal 40, the server terminal 10 receives it and additionally registers it in the authenticity determination support information database 142. Consequently, the authenticity determination support information database can be enhanced.

### Third Example Embodiment

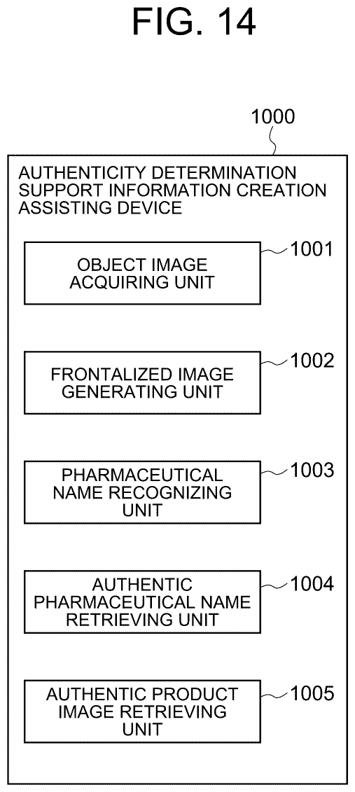

FIG. 14 is a block diagram of an assisting device 1000 according to a third example embodiment of the present invention.

Referring to FIG. 14, the assisting device 1000 includes an object image acquiring unit 1001, a frontalized image generating unit 1002, a pharmaceutical name recognizing unit 1003, a genuine pharmaceutical name retrieving unit 1004, and a genuine product image retrieving unit 1005.

The object image acquiring unit 1001 is configured to acquire an image obtained by capturing a surface showing the pharmaceutical name of a pharmaceutical package as an object image. The object image acquiring unit 1001 can be configured in the same manner as, for example, the object image acquiring unit 71 of FIG. 1, but is not limited thereto. The frontalized image generating unit 1002 is configured to generate a frontalized image by extracting a region of the above-described surface from the object image acquired by the object image acquiring unit 1001 and performing trapezoidal correction. The frontalized image generating unit 1002 can be configured in the same manner as, for example, the frontalized image generating unit 72 of FIG. 1, but is not limited thereto. The pharmaceutical name recognizing unit 1003 is configured to recognize the pharmaceutical name by performing character recognition from the frontalized image generated by the frontalized image generating unit 1002. The pharmaceutical name recognizing unit 1003 can be configured in the same manner as, for example, the pharmaceutical name recognizing unit 73 of FIG. 1, but is not limited thereto. The authentic pharmaceutical name retrieving unit 1004 is configured to retrieve a similar pharmaceutical name from the authorized pharmaceutical name list by using the pharmaceutical name recognized by the pharmaceutical name recognizing unit 1003, as authentic pharmaceutical name. The authentic pharmaceutical name retrieving unit 1004 can be configured in the same manner as, for example, the authentic pharmaceutical name retrieving unit 74 of FIG. 1, but is not limited thereto. The authentic product image retrieving unit 1005 is configured to retrieve an authentic product image from image information in which the authentic pharmaceutical name and the authentic product image obtained by capturing a surface showing the pharmaceutical name of the authentic pharmaceutical package are associated with each other, by performing image retrieval using the authentic pharmaceutical name retrieved by the authentic pharmaceutical name retrieving unit 1004 as a keyword. The authentic product image retrieving unit 1005 can be configured in the same manner as, for example, the authentic product image retrieving unit 75 of FIG. 1, but is not limited thereto.

The assisting device 1000 configured as described above operates as described below. That is, the object image acquiring unit 1001 acquires an image obtained by capturing a surface showing the pharmaceutical name of a pharmaceutical package as an object image. Then, the frontalized image generating unit 1002 generates a frontalized image by extracting a region of the above-described surface from the object image acquired by the object image acquiring unit 1001 and performing trapezoidal correction. Then, the pharmaceutical name recognizing unit 1003 recognizes the pharmaceutical name by performing character recognition on the frontalized image generated by the frontalized image generating unit 1002. Then, the authentic pharmaceutical name retrieving unit 1004 retrieves a similar pharmaceutical name from the authorized pharmaceutical name list by using the pharmaceutical name recognized by the pharmaceutical name recognizing unit 1003 as the authentic pharmaceutical name. Then, the authentic product image retrieving unit 1005 retrieves an authentic product image from image information in which the authentic product name and the authentic product image obtained by capturing a surface showing the pharmaceutical name of the authentic pharmaceutical package are associated with each other, by performing image retrieval using the authentic pharmaceutical name retrieved by the authentic pharmaceutical name retrieving unit 1004 as a keyword.

By using the assisting device 1 that is configured and operates as described, it is possible to reduce the time and cost when an expert creates information to support an authenticity determination of a pharmaceutical product by a user. This is because the expert only needs to confirm the recognized pharmaceutical name and does not need to manually enter the pharmaceutical name by visually reading it from the object image. Further, when the shape deformation of the object image is large, it may be difficult to apply character recognition, or the possibility of misrecognition may increase, but since character recognition is performed after trapezoidal correction is performed by the frontalized image generating unit 1002, character recognition can be performed with high accuracy without such problems. Since the authentic pharmaceutical name retrieving unit 1004 is provided, the expert can save the trouble of searching for whether or not it is an authorized pharmaceutical product. In addition, since the authentic product image retrieving unit 1005 is provided, the expert can save the trouble of performing image retrieval and acquiring an authentic product image by himself/herself. As described above, the expert can save the trouble of determining whether it is an authorized pharmaceutical product or retrieving an authentic product image by himself/herself, so that the expert can take sufficient time to determine whether the object image is an image of a genuine package or a fake. In addition, it is possible to refer to whether it is an authorized pharmaceutical product or the content of a genuine product image, for determination information.

Although the present invention has been described above with reference to the above example embodiments, the present invention is not limited to the example embodiments described above. The configuration and details of the present invention can be changed in various manners that can be understood by those skilled in the art within the scope of the present invention.

For example, a GPU (Graphic Processing Unit), a DSP (Digital Signal Processor), an MPU (Micro Processing Unit), an FPU (Floating Number Processing Unit), a PPU (Physics Processing Unit), a TPU (Tensor Processing Unit), a quantum processor, a microcontroller, or a combination of these may be used, instead of the aforementioned CPU.

### INDUSTRIAL APPLICABILITY

The present invention is applicable to the field of determining authenticity of a pharmaceutical package.

### REFERENCE SIGNS LIST

1 authenticity determination support information creation assisting device
2 camera
3 communication I/F unit
4 operation input unit
5 screen display unit
6 storing unit
7 arithmetic processing unit

What is claimed is:

1. An authenticity determination support information creation assisting device comprising:
a memory containing program instructions; and
a processor coupled to the memory, wherein the processor is configured to execute the program instructions to:
acquire an image obtained by capturing a surface showing a pharmaceutical name of a pharmaceutical package as an object image;
generate a frontalized image by extracting a region of the surface from the acquired object image and performing trapezoidal correction;
recognize the pharmaceutical name by performing character recognition from the generated frontalized image;
retrieve a similar pharmaceutical name as an authentic pharmaceutical name from an authorized pharmaceutical name list by using the recognized pharmaceutical name; and
retrieve an authentic product image from image information in which the authentic pharmaceutical name and the authentic product image obtained by capturing a surface showing a pharmaceutical name of an authentic pharmaceutical package are associated with each other, by performing image retrieval using the retrieved authentic pharmaceutical name as a keyword.

2. The authenticity determination support information creation assisting device according to claim 1, wherein the processor is further configured to execute the program instructions to
detect a region having a large difference between the generated frontalized image and the retrieved authentic product image.

3. The authenticity determination support information creation assisting device according to claim 2, wherein
the detecting the region having the large difference includes detecting the region having the large difference by using a trained model in which machine learning has been performed for estimating the region having the large difference between the frontalized image and the authentic product image.

4. The authenticity determination support information creation assisting device according to claim 1, wherein
the generating the frontalized image includes:
detecting an edge of a package shown in the object image;
detecting a surface of the package shown in the object image;
applying watershed that separates an object such as a package from a background in the object image;
outputting a surface image of the package shown in the object image, from the object image, a detection result of the edge, a detection result of the surface, and a separation result of the object and the background
detecting an outer contour of the surface image; and
a performing trapezoidal correction on a shape of a surface of the package specified by the outer contour, and generating the frontalized image.

5. The authenticity determination support information creation assisting device according to claim 1, wherein
the recognizing the pharmaceutical name includes
calculating a character recognition result of one or more character strings shown in the frontalized image and a circumscribed area, and outputting the character recognition result in which prioritization is performed in accordance with a size of the circumscribed area.

6. An authenticity determination support information creation assisting method comprising:
acquiring an image obtained by capturing a surface showing a pharmaceutical name of a pharmaceutical package as an object image;
generating a frontalized image by extracting a region of the surface from the acquired object image and performing trapezoidal correction;
recognizing a pharmaceutical name by performing character recognition from the generated frontalized image;
retrieving a similar pharmaceutical name as an authentic pharmaceutical name from an authorized pharmaceutical list by using the recognized pharmaceutical name; and
retrieving an authentic product image from image information in which the authentic pharmaceutical name and the authentic product image obtained by capturing a surface showing a pharmaceutical name of an authentic pharmaceutical package are associated with each other, by performing image retrieval using the retrieved authentic pharmaceutical name as a keyword.

7. A non-transitory computer-readable medium storing thereon a program comprising instructions for causing a computer to execute processing to:
acquire an image obtained by capturing a surface showing a pharmaceutical name of a pharmaceutical package as an object image;
generate a frontalized image by extracting a region of the surface from the acquired object image and performing trapezoidal correction;
recognize a pharmaceutical name by performing character recognition from the generated frontalized image;
retrieve a similar pharmaceutical name as an authentic pharmaceutical name from an authorized pharmaceutical list by using the recognized pharmaceutical name; and
retrieve an authentic product image from image information in which the authentic pharmaceutical name and the authentic product image obtained by capturing a surface showing a pharmaceutical name of an authentic pharmaceutical package are associated with each other, by performing image retrieval using the retrieved authentic pharmaceutical name as a keyword.

8. An authenticity determination support system for supporting authenticity determination on a determination object by a user, the system comprising:
a database that is created by using the authenticity determination support information creation assisting device according to any claim 1 and in which authenticity determination support information is stored, the authenticity determination support information including an image obtained by capturing a genuine pharmaceutical package and an image obtained by capturing a fake of the genuine pharmaceutical package;
a memory containing program instructions; and
a processor coupled to the memory, wherein the processor is configured to execute the program instructions to:
acquire an image obtained by capturing the determination object from a user terminal;
match the image obtained by capturing the determination object against an image in the authenticity determination support information stored in the database; and
when the authenticity determination support information including an image matching the image obtained by capturing the determination object is detected as a result of the matching, allow the detected authenticity determination support information to be displayed on a screen of the user terminal.
